(19) United States
(12) Patent Application Publication
(10) Pub. No.: US 2002/0037516 A1
(43) Pub. Date: Mar. 28, 2002

(54) Assay for identification of a test compound

(76) Inventors: Alastair Murchie, Cambridge (GB); Georg Lentzen, Cambridge (GB)

Correspondence Address: PALMER & DODGE, LLP, ONE BEACON STREET, BOSTON, MA, 02108-3190, US

(21) Appl. No.: 09/839,649

(22) Filed: Apr. 19, 2001

Related U.S. Application Data
(60) Provisional application No. 60/198,179, filed on Apr. 19, 2000.

(51) Int. Cl. C12Q 1/68; C12P 19/34

(52) U.S. Cl. 435/6; 435/91.2

(57) ABSTRACT

A method for determining whether a test compound binds to a target RNA, the method comprising the steps of: (a) contacting the test compound with the target RNA and a RNA-modifying enzyme; and (b) detecting the modification of the target RNA by the enzyme and comparing the amount of modification detected to that of a standard. Inhibition of enzymatic modification of the RNA target indicates that the test compound can bind the target RNA and can thus exhibit antibiotic activity.

This application claims the priority of U.S. Provisional Patent Application No. 60/198,179, filed Apr. 19, 2000, the entirety of which is incorporated herein by reference, including figures.

All documents cited herein are incorporated by reference in their entirety.

### TECHNICAL FIELD OF THE INVENTION

This invention is in the field of assays for antibiotics and other low molecular weight RNA binding compounds, more particularly assays for identifying compounds that inhibit enzymatic modifications of RNA which result in antibiotic resistance and RNA maturation.

### BACKGROUND OF THE INVENTION

Problems of antibiotic resistance are well documented, and new antibiotics are needed.

Many antibiotics function by inhibiting protein synthesis, typically by acting at the level of rRNA. The rRNA binding sites of many different types of antibiotics have been mapped by chemical and enzymatic probing to various sub-regions on the 16S and 23S rRNAs. Examples of antibiotics targeted to these sites include binding of the 16S rRNA A site by members of the aminoglycoside class, binding of the 23S rRNA L1 site (the E site) by the oxazolidinone class, and binding of the 23S rRNA GTPase center by the thiazole class [Spahn & Prescott, 1996].

In some cases, modification of the RNA target of an antibiotic (e.g. by methylation) can result in resistance [Cundliffe, 1978; Skinner et al., 1983; Clancy et al., 1995]. For example, methylation of A-1067 in the 23S RNA of Streptomyces azureus results in thiostrepton resistance, and mono- or di-methylation of the N6 position of A-2058 results in erythromycin resistance.

It is an object of the present invention to provide methods for identifying compounds that bind to RNA and thereby inhibit RNA modifying enzymes, including enzymes which introduce modifications that induce resistance to established antibiotics.

### SUMMARY OF THE INVENTION

The invention provides a method for determining whether a test compound binds to a target RNA, the method comprising the steps of: (a) contacting the test compound with the target RNA and a RNA-modifying enzyme; and (b) detecting the modification of the target RNA by the enzyme and comparing the amount of modification detected to that of a standard, wherein the comparing determines whether the test compound binds to the target RNA.

In one embodiment, the target RNA comprises a rRNA or a fragment or sub-region thereof.

In another embodiment, the target RNA comprises a whole ribosome.

In another embodiment, the target RNA is a ribosome fragment or sub-region thereof.

In another embodiment, the target RNA includes a stabilising structure.

In another embodiment, the target RNA comprise a chemical modification which enhances the stability of the target RNA.

In another embodiment, the RNA-modifying enzyme is selected from the group consisting of a methyltransferase, a pseudouridine synthase, a guanine glycosylase, a G37-N1-methylguanosine-tRNA-methyltransferase, and a 2′-O-ribosyl phosphate transferase.

In a preferred embodiment the methytransferase is the thiostrepton resistance methyltransferase or the erythromycin resistance methyltransferase. It is further preferred that target RNA modification is detected by the incorporation of an isotopic label from S-adenosyl-methionine into the target RNA.

In another embodiment, the test compound is selected from the group consisting of a peptide, a peptoid, a protein, a lipid, a metal, a nucleotide, a nucleoside, a small organic molecule, and a polyamine.

In another embodiment, the test compound is selected from a combinatorial library.

In another embodiment, the method is performed in a high-throughput screening format.

The invention further encompasses a test compound that binds to a target RNA, the test compound identified by the method of claim 1.

The invention further encompasses a kit for determining whether a test compound binds to a target RNA, the kit comprising the target RNA and a RNA-modifying enzyme.

The invention further encompasses a method for determining whether a test compound binds to a target RNA, the method comprising the steps of: (a) contacting the test compound with a RNA-modifying enzyme and the target RNA, wherein the target RNA comprises a suicide substrate for the enzyme; and (b) detecting the modification of the enzyme by the suicide substrate, wherein the detecting determines whether the test compound binds to the target RNA.

### BRIEF DESCRIPTION OF THE FIGURES

FIG. 1 shows various RNA modifications.

FIG. 2 shows the structural elements of the thiostrepton binding site in the GAR RNA of 23S RNA.

FIG. 3 shows the results of a methylation experiment carried out around the thiostrepton binding site in the GAR RNA of 23S RNA.

FIG. 4 shows the results of testing four additional substrates for methyltransferase. FIG. 4A shows testing of the human mGAR (H.s. GAR), a left-hand loop 17-mer fragment of E. coli mGAR (LHL), a left hand loop 29-mer of E. coli mGAR, and the E. coli 60-mer complete mGAR. FIG. 4B shows a binding curve for two of the reactions in FIG. 4A (complete E. coli mGAR and E. coli 29-mer GAR).

FIG. 5 shows the titration of L 11 protein in the presence of methyltransferase.

FIG. 6 shows the titration of thiostrepton in the presence of methyltransferase.

FIG. 7 shows binding curves for 4 different test compounds.

FIG. 8 shows the site of action of the erythromycin resistance methyltransferase (erm) in the peptidyl transferase centre. The enzyme dimethylates N6 of A2058 of 23S rRNA. FIG. 9 shows the positions of methylation modifications in the decoding site of 16S rRNA that confer resistance to aminoglycoside antibiotics. FIG. 10 shows RNA sequences of the thiostrepton binding fragment for a range of bacteria.

FIG. 11 shows the decoding sites of 16S rRNA for a range of bacteria. FIG. 12 shows uridine (grey) residues in 16S rRNA modified by pseudouridine synthase. FIG. 13 shows uridine residues (grey) modified by pseudouridine synthase for the 5′ half of 23S rRNA

FIG. 14 shows uridine residues (grey) modified by pseudouridine synthase for the 3′ half of 23S rRNA.

FIG. 15 shows recovery of GAR RNA methylation activity in the presence of L11 by deletion of the N terminal domain of L11.

FIG. 16 shows the reproducibility of TSR methylase assay.

FIG. 17 shows interassay variation of the GAR methylation single point assay in a 96-well format. The graph shows the results of two independent assays of the same set of 80 compounds, measured in a single point assay at 50 μM, plotted against each other.

FIG. 18 shows methylation of rRNA by Erm methylase.

FIG. 19 shows the effect of refolding on rRNA methylation by GST-Erm.

FIG. 20 shows magnesium dependence of rRNA methylation by ERM.

FIG. 21 shows the decrease in refolded rRNA methylation by ErmE in the presence of erythromycin.

FIG. 22 shows inhibition of rRNA methylation with ERM by various compounds

### DETAILED DESCRIPTION OF THE INVENTION

The invention provides a method for determining whether a test compound binds to a target RNA, the method comprising the steps of:

(a) contacting the test compound with the target RNA and a RNA-modifying enzyme; and

(b) measuring the modification of the target RNA by the enzyme and comparing the measured value to that of a standard.

Inhibition of enzymatic modification of the RNA target indicates that the test compound is capable of binding to the RNA and can thus exhibit antibiotic activity.

The Target RNA

The target RNA can comprise any RNA which is a substrate of and can be modified by the RNA-modifying enzyme. Preferably, it is a RNA which can be bound by a known antibiotic and, more preferably, one which is known to be modified in vivo to confer resistance to that antibiotic.

The target RNA can be derived from fungal, viral, bacterial (Gram-negative or -positive), or eukaryotic RNA. It can be from a natural source, can be an enzymatically transcribed RNA, or can be synthetic in whole or in part.

Typically, the target RNA comprises a rRNA (e.g. a 16S or 23S rRNA), or a fragment or sub-region thereof. As used herein, the term ‘fragment or sub-region thereof’ refers to a RNA that comprises a substrate site for a RNA modifying enzyme but comprises fewer than all of the nucleotides of the RNA comprising that sequence as it sis expressed in nature. A fragment of sub-region of a target RNA can additionally comprise stabilizing sequences that permit the fragmentor sub-region to adopt an essentially native conformation. Non-limiting examples of suitable target RNAs include the following rRNA sub-regions: the A site in 16S rRNA [Fourmy et al., 1996; Holmes & Cundliffe, 1991]; the spectinomycin site in 16S rRNA [Brink et al., 1994]; the peptidyltransferase centre in 23S rRNA [Kovalic et al., 1995]; the L1 binding site (the E site) in 23S rRNA [Matassova et al., 1999]; the minimal thiostrepton binding fragment of 23S rRNA, also known as the GTPase activating region (GAR) [Ryan et al., 1991]; a 16S fragment which retains aminoglycoside binding [e.g. Purohit and Stem, 1994; see also U.S. Pat. No. 5,712,096].

As used herein, the term “essentially native conformation” refers to a conformation of a target RNA that permits the target RNA to be a substrate for the RNA modifying enzyme. A target RNA with an essentially native conformation is modified by a given RNA modifying enzyme at the same site or sites and in the same manner as a native RNA comprising that target RNA sequence or sequences. One of skill in the art can determine if a target RNA adopts an essentially native conformation by exposing the target RNA to the RNA modifying enzyme and detecting modification. If modification, e.g., methylation, occurs on the target RNA at the same site or sites at which a native RNA comprising those target sequences is modified, the target RNA has an essentially native conformation.

As an alternative to using rRNA, the invention can be used to target other types of RNA. Non-limiting examples of suitable target RNAs in this category include the HV-1 RRE transcriptional activator region [Zapp et al., 1993], self-splicing group I intron RNA [von Ahsen et al., 1991], mRNA [Bokar et al., 1994], tRNA [Grosjean et al., 1995], tmRNA [Felden et al., 1998], RNase P RNA and ribozymes [Stage et al., 1995]

Other suitable target RNAs include, for example, whole ribosomes, ribosomal subunits, signal recognition particle (SRP) RNA, viroid RNA and RNA viruses. Mimics of these RNAs can also be used.

Target RNA sequences for use in the present invention are typically between 5 and about 3000 nucleotides in length, preferably between 20-100 nucleotides, and most preferably 30-80 nucleotides. For example, the target RNA can comprise a chemically-synthesised oligonucleotide of between 20 and 100 nucleotides in length that is capable of folding to form a secondary structure present in the native rRNA.

The target RNA can be formed from a single polynucleotide, folded back on itself to form secondary and tertiary structure, or can be formed from two or more annealed polynucleotides which interact to form secondary and tertiary structure. Examples of the use of two or more oligonucleotides which, after annealing, form a folded target RNA target are given in Karn et al. [WO92/05195; U.S. Pat. No. 5,786,145], describing mimics of the RRE. Synthetic analogues of ribozymes formed by annealing oligonucleotides have also been described [Grasby et al., 1993; Slim et al., 1991].

As well as comprising sequences which form the binding site of the RNA-modifying enzyme, the target RNA can include stabilising or connecting structures (e.g. double-stranded regions, tetraloops, stem-loop structures etc.) added to the target RNA sequence. A stabilising structure is a structure comprised by a sequence or sequences, not normally associated with the target RNA sequence in nature, which permits or assists the target RNA to assume and maintain an essentially native conformation. Examples include, but are not limited to double-stranded regions, tetraloops, and stem-loop and hairpin structures. Connecting structures are regions of complementary nucleotide sequence that hybridize to connect two separate RNAs or two regions of an RNA molecule in order to generate all or part of a target RNA modifying enzyme substrate site. The stability of the target RNA conformation can also be increased by including in the reaction mixture an RNA-binding protein, e.g. a ribosomal protein that is usually associated with the complete RNA substrate.

As RNA is sensitive to cleavage by cellular ribonucleases, as well as to alkaline or acid conditions, it can be desirable to modify the target RNA to enhance its stability against degradation, or to provide functional groups for immobilising the RNA on solid supports by covalent or non-covalent attachments. As used in this context, the term “chemical modification” means a covalent modification (including addition, removal or substitution of chemical groups) of an RNA molecule, in the base, sugar, and/or phosphodiester linkages between sugar moieties that protects the RNA from one or more degrading forces or functionalizes it for an assay format. While it is acknowledged that in the common usage a covalent enzymatic modification is a “chemical modification”, as used herein, “chemical modification” is not accomplished with the aid of an enzyme or is accomplished as a discrete and separate step.

Chemical modifications of the target RNA include, but are not limited to, the following types:

a) Backbone modifications:

(i) phosphorothioates (single S substituents or any combination of two or more with the remainder as O; (ii) methylphosphonates; (iii) phosphoramidates; (iv) phosphotriesters; (v) phosphorus-free linkages (e.g. carbamate, acetamidate, acetate)

b) Sugar modifications:

(i) 2′-deoxynucleosides (R=H); (ii) 2′-O-methylated nucleosides (R=OMe); (iii) 2′-fluoro-2′-deoxynucleosides (R=F); (iv) 2′-O-alkylated nucleosides

c) Base modifications:

(i) pyrimidine derivatives substituted in the 5-position (e.g. methyl, bromo, fluoro etc . . . or replacing a carbonyl group by an amino group); (ii) purine derivatives lacking specific nitrogen atoms (e.g. 7-deaza-adenine, hypoxanthine) or functionalised in the 8-position (e.g. 8-azido adenine, 8-bromo adenine), or additional functionalities (e.g. 2,6-diaminopurine)

d) Oligonucleotides covalently linked to reactive functional groups (e.g. psoralens, phenanthrolines, mustards)

e) irreversible cross-linking agents with or without the need for co-reagents)

(i) acridine (intercalating agents); (ii) thiol derivatives (reversible disulphide formation with proteins); (iii) aldehydes (Schiff's base formation); (iv) azido, bromo groups (UV cross-linking); (v) ellipticenes (photolytic cross-linking)

f) oligonucleotides containing haptens or other binding groups;

g) fluorescent moieties or other non-radioactive labels; and

h) combination of two or more modifications selected from a) to g)

Further details of modifications can be found in, for instance, Gait et al. [pages 1-36 of RNA-Protein Interactions: A Practical Approach (ed. Smith) Oxford University Press (1998)].

As used herein, the term “enhanced stability” means that the half-life of the target RNA is at least 10% longer, and preferably 20% longer, 50% longer, 100% longer, 3-fold longer, or 5-fold, 10-fold, 50-fold, or even 100-fold or more longer for a target RNA comprising a chemical modification than for a target RNA of the same sequence that lacks the modification. RNA half-life is measured, for example, by gel electrophoresis of labelled RNA isolated after various times of incubation under potentially degrading conditions.

Binding of an RNA-associated protein can inhibit the activity of the RNA-modifying enzyme. RNA-modifying activity can in some cases be recovered by deletion of one or more domains of the RNA protein. It is therefore possible to modify the assay such that RNA-associated proteins or fragments of such proteins are present. In an embodiment of the present invention, the RNA-associated proteins or domains thereof, which inhibit the activity of the RNA-modifying enzyme are absent, while RNA-associated proteins or domains thereof required for or beneficial to maintaining the native conformation of the RNA for recognition by the RNA-modifying enzyme, are present.

The RNA-modifying Enzyme

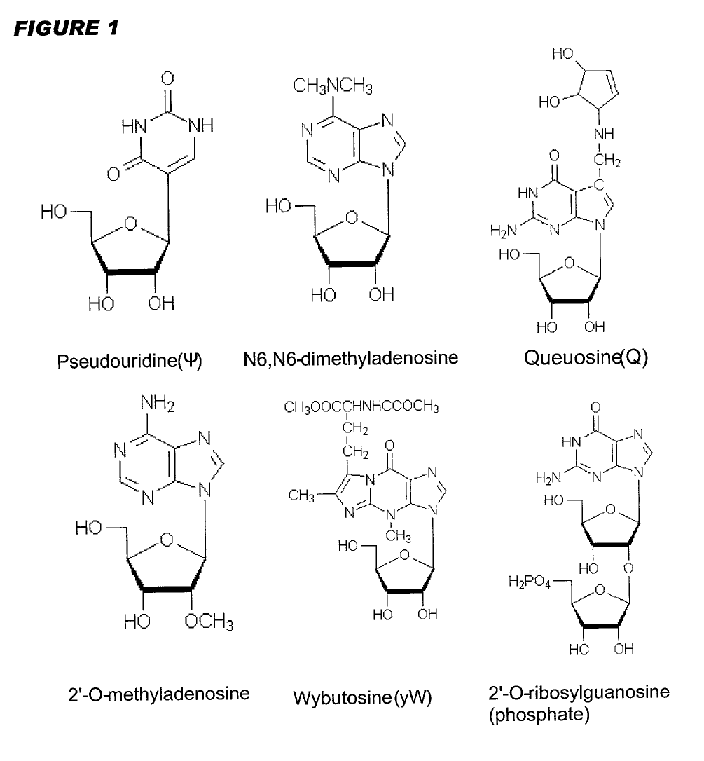

A number of enzymes that covalently modify RNA are known. The modification typically involves the covalent addition to, or alteration of, existing bases in RNA [Limb ach et al., 1994; Rozenski et al., 1999]. Modifications usually take place at specific positions i.e. not all bases are modified. Some modifications are shown in FIG. 1.

The most common form of RNA modification is methylation, in which a methyl group is transferred by a methyltransferase enzyme from S-adenosyl-methionine (SAM) to a position on the RNA. Methyl groups can be introduced at various positions on the bases and also at the 2′-OH position of the ribose. Methyltransferases are also responsible for dimethylation. Methyltransferases include, for example: the E. coli 23S rRNA methyltransferase RrmJ/FTSJ (Caldas et al., 2000, J. Biol. Chem. 275: 16414-16419); the E. coli 16S rRNA m5C967 methyltransferase (Gu et al., 1999, Biochemistry 38: 4053-4057); vaccinia virus VP39 2′-O-methyltransferase (Lockless et al., 1998, Biochemistry 37: 8564-8574); Irm, encoding a 26 kDa ribosomal RNA methyltransferase that confers high-level resistance to lincomycin with lower levels of resistance to macrolides (Jenkins et al., 1991, Gene 108: 55-62); the Micromonospora. purpurea grm protein, a ribosomal RNA methyltransferase; the ribosomal RNA N-methyltransferase that confers self-resistance on the erythromycin-producing bacterium Saccharopolyspora erythraea (Dhillon et al., 1990, FEBS Lett. 262: 189-193); the erythromycin methyltransferase of an erythromycin-producing species of Arthrobacter (Dhillon et al., 1990, supra); carB methyltransferase from Streptomyces griseofuscus (Epp et al., 1987, Gene 53: 73-83); and ermE methyltransferase from Streptomyces erythraeus (Epp et al., 1987, supra).

Pseudouridine synthase reorients an existing uridine to pseudouridine (Ψ) in tRNAs and rRNAs of bacteria and eukaryotes [Foster et al, 2000; Ganot et al., 1997]. Examples of pseudouridine synthase include, but are not limited to: E. coli truB (Gutgsell et al., 2000, RNA 6: 1870-1881); Schizosaccharomyces pombe Pus1p (Hellmuth et al., Nucl. Acids res. 28: 4604-4610); E. coli Rl UD (Wrzesinski et al., 2000, IUBMB Life, 50: 33-37); and 23S RNA pseudouridine 2633 synthase from B. subtilis. Queuosine can replace guanine in tRNA and is incorporated by the enzyme tRNA guanine glycosylase in both bacteria and eukaryotes. Wyosine is made by the multi-step modification of an existing guanine or inosine, proceeding via N1-methylguanosine [Droogmans & Grosjean, 1987] using a G37-N1-methylguanosine-tRNA-methyltransferase. The enzyme 2′-O-ribosyl phosphate transferase introduces a ribose at the 2′ position of a guanine in eukaryotic tRNA (e.g. RitI [Astrom & Bystrom, 1994]). A summary of modifications is given in Table I. Acetylases are involved in N4-acetylcytidine, N4-acetyl-2′-O-methylcytidine modifications. ThiI and IscS are involved in 4-thiouridine biosynthesis. Aminoacyl-tRNA-synthetases are riot considered RNA modifying enzymes according to the invention.

In the methods of the invention, therefore, preferred RNA-modifying enzymes include, but are not limited to methyltransferases (e.g. thiostrepton resistance methyltransferase, erythromycin resistance methyltransferase, G37-N1-methylguanosine-tRNA-methyltransferases), pseudouridine synthases (e.g. pseudouridine synthase I), guanine glycosylases, methylguanosine-tRNA-methyltransferases, 2′-O-ribosyl phosphate transferases (e.g. RitI), acetylases, ThiI and IscS. These can be of any origin, prokaryotic, archaeal or eukaryotic.

Functional fragments of RNA modifying enzymes can also be used, as can allelic variants and mutants.

Enzymes used in the invention are preferably in an essentially pure state, but the invention can also utilise impure enzyme or a cell extract.

RNA-modifying enzymes have very stringent structural or sequence requirements for the RNA substrate. Using conventional techniques of protein engineering, therefore, it is possible to modify a known enzyme to alter its substrate preference. In this way, an available enzyme can be tailored to modify a target RNA of interest, for use in the methods of the invention.

The modification of new sites on a target RNA molecule can also be achieved by utilising a small nucleolar ribonucleolar protein complex (snoRNP), an RNA modifying activity [Samarsky et al. 1998; Ganot et al., 1997; Kiss-Laszlo et al., 1996; U.S. Pat. No. 5,989,911] that exploits sequence complementarity within the snoRNA as a guide to the complementary sequence on the target rRNA. There are two large families of RNA-modifying snoRNP.

One snoRNP family is associated with a 2′-O-methylation activity and is involved in the maturation of eukaryotic pre-rRNA [Kiss-Laszlo et al., 1996]. The complex contains a number of protein components that are responsible for the methylation activity including the essential nucleolar protein fibrillarin. The RNA component consists of an RNA guide sequence that has sequence complementarity (10-21 nucleotides) to the target rRNA and two sequence motifs: box C at the 5′ end of the snoRNA molecule and D at the 3′ end, box D (or D′) is positioned five base pairs from the methylated nucleotide. Mutations in this region of the snoRNA that introduce sequence complementarity to a target RNA of interest, would make the target RNA accessible for use in the methods of the invention.

The other rRNA modifying snoRNP family is associated with a pseudouridilation activity and is also involved in the maturation of eukaryotic pre-rRNA [Samarsky et al., 1998]. A pseudouridine synthase is responsible for the pseudouridilation activity. The sno RNA component that acts as a guide sequence has a hairpin-hinge-hairpin-tail secondary structure and two essential sequence elements: the 5′ hinge region (box H), has a consensus sequence ANANNA and the 3′ tail region (box ACA) has a consensus sequence ACA but can include the sequences AUA and AAA. Pseudouridine formation takes place in pre-rRNA at regions that are homologous to boxes ACA and box H but at positions within the pre-rRNA that are complementary to box H. Sequence changes within box H and box ACA that introduce sequence complementarity to a target RNA of interest, would make the target RNA accessible for use in the methods of the invention.

Suitable enzymes can also been identified by data-mining genomic sequences, based on the nucleotide sequence and structure characteristics of known enzymes [e.g. Klimasauskas et al., 1989; Schluckebier et al., 1995].

TABLE I
Enzymatic RNA Modifications and Their Detection

| Modification | Enzyme | Modified site | Detection | Ref |
|---|---|---|---|---|
| 2’-O methylation | Thiostrepton resistance | Bacterial 23S rRNA A1067 | ³H-SAM; | 1 |
| | methyltransferase | | absence of band on | |
| | sno methylase | Eukaryotic rRNA | sequencing gel | 2 |
| N6 A | Erythromycin resistance | Bacterial 23S rRNA A2058 | | 3 |
| dimethylation | methylase | | | |
| N6 A | ksgA methylase | Bacterial and eulkaryotic 16S, | | 4 |
| dimethylation | | 18S rRNA A1518 A1519 | | |
| N1 A methylation | kamC resistance methylase | Bacterial 16S rRNA A1408 (A | | 5 |
| | | site) | | |
| N7 G methylation | kgmB methylase | Bacterial 16S rRNA G1405 (A | | 6 |
| | | site) | | |
| Pseudouridine | Pseudouridine synchase | tRNA sites; 16S & 23S rRNA | Mass spectrometry; | 7, 8 |
| (Ψ) | | in archaea and bacteria; 18S | absence of band on | |
| | | & 28S rRNA in eukarya | sequencing gel - | |
| | | | chemical modification | |
| | | | by CMC* detected on | |
| | | | gel by preventing | |
| | | | extension by reverse | |
| | | | transcriptase; | |
| | | | incorporation of ³²P-5- | |
| | | | FUTP | |
| Queuosine (Q) | tRNA-guanine transglycosylase | tRNA sites in bacteria and | Radiolabelled | 9 |
| | (Tgt): | eukarya | 7-aminomethyl-7- | 10 |
| | S-adenosylmethionine: tRNA | | deazaguanine; | |
| | ribosyltransferase-isomerase | | Radiolabelled SAM | |
| | (QueA) | | | |
| Wyosine (Y) | G37-N1-methyltransferase | tRNA sites in eukarya | Characteristic | 11 |
| | and other enzymes | | fluorescence signal | |
| 2’-O ribosylation | 2’-O-ribosyl phosphate | tRNA sites in eukarya | Mass spectrometry; | 12 |
| | transferase | | absence of band on | |
| | | | sequencing gel | |

*1-cyclohexyl-3-(2-morpholinoethyl) carbodiimide metho-p-toluene sulfonate (CMC)
Table I references:
1. Thompson & Cundliffe, 1981
2. Kiss-Laszlo et al., 1996
3. Skinner et al., 1983
4. van Buul & van Knippenberg, 1985
5. Holmes et al., 1991
6. Holmes & Cundliffe, 1991
7. Foster et al., 2000
8. Huang et al., 1998
9. Janel et al., 1984
10. Slany et al., 1994
11. Droogmans & Grosjean, 1987
12. Astrom & Bystrom, 1994

Measurable Changes

The methods of the invention involve the detection and/or measurement of target RNA modification by the RNA-modifying enzyme (i.e. an indirect measurement of the interaction of the test compound and the RNA target). As used herein, the term “detecting the modification of target RNA” refers to the process whereby one determines whether a particular RNA modifying enzyme has covalently modified the target RNA. The detection and/or measurement of target RNA modification can be performed in a variety of ways, depending upon the nature of the modification (see, for example, Table I).

The term “comparing the amount of modification detected to a standard” refers to the process whereby the amount of modification by a given RNA modifying enzyme is determined in separate reactions performed with and without a test compound. The modification detected in the reaction performed without the test compound is used as a standard for comparison with the amount detected in the presence of test compound.

Where the RNA-modifying enzyme is a methyltransferase, detection and/or measurement of modification is preferably based around the incorporation of an isotopic label from S-adenosyl-methionine (e.g. S-Adenosyl-L-[methyl-³H]Methionine=³H-SAM) into the target RNA.

In other cases, the modification can interfere with reverse transcriptase primer extension of a cDNA, or with a specific endonuclease. Radioactively labelled products of the primer extension reaction or end-labelled fragments of the target RNA can be analysed on a sequencing gel. The appearance or disappearance of a band, specific for a modification, indicates RNA modification. These can be quantified by scanning autoradiographs or phosphorimager analysis.

To obtain RNA from the reaction mixture for measuring modification, any standard RNA extraction protocol can be used. For example, RNA can be precipitated by the addition of TCA, TFA or ethanol solution. Precipitated RNA can then be isolated by centrifugation or by retention on filters (e.g. glass fibre, nitrocellulose, or other suitable filters).

As an alternative to detecting or measuring RNA modification in step (b), the methods of the invention can involve the detection or measurement of the modification of the RNA-modifying enzyme by the incorporation of a non-competitive, irreversible, suicide substrate from the target RNA. As used herein, the term “suicide substrate” refers to an enzyme substrate, e.g., a target RNA or a nucleotide or base within the target RNA, that when modified by the enzyme, irreversibly binds to and inhibits the further activity of the enzyme. Under these circumstances, one detects the activity of the enzyme on the target RNA by detecting a label incorporated into the target RNA that becomes bound to the enzyme. Where the RNA-modifying enzyme is a pseudouridine synthase, for instance, measurement of modification can be based around the incorporation of an isotopic label from 5-F uridine (e.g. α³²P-FUTP), a suicide substrate incorporated into the target RNA [Huang et al., 1998].

To obtain modified protein any standard protein extraction protocol can be used, e.g. protein can be isolated by retention on filters.

Test Compound

The present invention can be used to identify compounds capable of binding to any target RNA, preferably as part of a screening process.

As used herein, the term “test compound” refers to an agent comprising a compound, molecule, or complex, that is being tested for its ability to bind to a target RNA. Test compounds can be any agents including, but not restricted to, peptides, peptoids, proteins, lipids, metals, nucleotides, nucleosides, small organic molecules, polyamines, and combinations and derivatives thereof. Small organic molecules have a molecular weight between 50 and about 2,500 daltons, and most preferably in the range 200-800 daltons. Complex mixtures of substances, such as extracts containing natural products, or the products of mixed combinatorial syntheses, can also be tested and the component that binds to the target RNA can be purified from the mixture in a subsequent step.

Test compounds can be derived or selected from large libraries of synthetic or natural compounds. For instance, synthetic compound libraries are commercially available from Maybridge Chemical Co. (Trevillet, Cornwall, UK) or Aldrich (Milwaukee, Wis.). Alternatively, libraries of natural compounds in the form of bacterial, fungal, plant and animal extracts can be used. Additionally, test compounds can be synthetically produced using combinatorial chemistry either as individual compounds or as mixtures. A collection of compounds made using combinatorial chemistry is referred to herein as a combinatorial library.

A compound is said to have antibiotic activity if it slows the growth of a microorganism (doubling time at least twice as long as doubling time in the absence of the compound), halts the growth of a microorganism, or kills a microorganism. A test compound identified as an a candidate antibiotic using the methods of the invention can have antibiotic activity on its own, or it can be used in conjunction with another known antibiotic. While not wishing to be bound by any specific mechanism, a compound identified as a candidate antibiotic can function directly as an antibiotic by binding to an RNA and interfering with its function (e.g., binding to bacterial rRNA, preventing translation). Alternatively, the candidate antibiotic can bind to an RNA, preventing its modification by an enzyme that confers resistance to another antibiotic, and thereby rendering the microorganism sensitive to that other antibiotic. An example of this alternative mechanism is the binding of the compound to bacterial rRNA, which prevents the methylation of the rRNA, thereby rendering the bacterium sensitive to an antibiotic to which it was previously resistant.

The Standard

The methods of the invention involve a comparison of the RNA modifying enzyme activity with a standard.

Preferably, the standard is a control value measured in the absence of the test compound and is a property of the enzyme that can be used to measure its function.

It will be appreciated that the standard may have been determined before performing the method, or can be determined during or after the method has been performed. It can be an absolute standard.

Order of Mixing

In preferred embodiments of the invention, target RNA and test compound are mixed in a first step. Modification is started by adding the enzyme and its second substrate (e.g. SAM in the case of SAM-dependent methyltransferases). After incubation for a defined time, the enzymatic reaction is stopped (e.g. by precipitation of the RNA). The degree of modification is compared with a standard. A reduction in modification compared with the standard indicates that the test compound and RNA form a strong complex.

As used herein, the term “reduction in modification” or “inhibition of modification” means that the amount of modification of the target RNA (or, when a suicide substrate is used, the enzyme itself) is at least 10% lower, and preferably 20% lower, 30% lower, 40% lower, 50% lower, 60% lower, 70% lower, 80% lower, 90% lower, 95% lower, 97% lower, 99% lower, or even up to and including 100% lower (i.e., no modification) in the presence of a test compound than in the absence of that test compound.

The mixture can also include a known antibiotic, in order to measure the ability of a test compound to displace a bound antibiotic. Compounds that displace antibiotics but do not inhibit RNA modification will lead to an increase in modification compared with the standard.

Some test compounds can be enzyme inhibitors without also being able to bind to the target RNA. These would be identified as false positives in an assay where simple inhibition of modification is measured. In order to avoid these false positives, the test compounds are preferably also measured against a range of enzymes (e.g. where the enzyme is a methyltransferase, against O-catechol methyltransferases, or restriction enzyme methyltransferases) in order to distinguish general enzyme inhibition from the inhibition of specific target RNA modification. As an alternative control, test compounds can be tested for their ability to displace pre-bound antibiotics. The reaction mixture can contain competitor RNA and DNA to increase the stringency of binding conditions and specificity of inhibition.

Quantitative Nature of Assay

An important feature of the invention is that the test compound competes for RNA binding with the enzyme. This provides specificity in the assay and permits exclusion of compounds that bind to the target RNA but do not interfere with RNA modifying enzymes.

The Kₘ for each titration is determined by fitting the data to the equation

y=Bₘₐₓ×[RNA]/(Kₘ+[RNA])

with

Bₘₐₓ: incorporation at RNA substrate saturation in cpm.

[RNA]: concentration of target RNA.

Kₘ: Michaelis constant

The Kᵢ value for a compound is determined by relating the Kₘ in the presence and the Kₘ in the absence of compound using the equation:

Kᵢ=[I]/((Kₘ(I)/Kₘ)−1)

with

[I]: concentration of the inhibitory compound

Kₘ(I): Michaelis constant in the presence of inhibitor

Kₘ: Michaelis constant in the absence of inhibitor

In an preferred format, enzyme binding and activity is measured by measuring the Michealis constant (Kₘ) of the enzymatic reaction in the absence and presence of a constant concentration of the test compound. Increasing amounts of target RNA are incubated with the modifying enzyme and the yield of modified RNA product is measured for each point. Data from this substrate titration are fitted to a saturation curve by non-linear regression. The Michaelis constant Kₘ is determined from the curve-fitting by routine methods.

In an alternative method, modification is quantitated at a constant target RNA concentration and a constant concentration of test compound. Measurements can be performed in duplicate or multiple and compared to a standard without compound (single point measurement).

Library Screening (Including High Throughput Screens)

The method can be used in the identification of compounds that bind to the target RNA from within a plurality of test compounds, such as in screening methods. The method can therefore involve the initial step of providing a plurality of test compounds, which can include compounds not already known to bind to the target RNA.

As used herein, the term “high throughput screening format” refers to a screening format with the capacity to test large numbers of compounds efficiently, typically implying that a number of compounds are tested simultaneously. For example, using 96-well microtiter plates, up to 96 compounds can be tested at a time. In addition, the capacity can be further increased by using more than one 96 well plate (e.g., 2, 5, 10, 50, 100, 200 or more plates). A high throughput screening format also has the capacity to simultaneously test a range of different concentrations of one or more test compounds.

For preferred high-throughput screening methods, all the biochemical steps for this assay are performed in a single solution in, for instance, a test tube or microtitre plate, and the test compounds are analysed initially at a single compound concentration. For the purposes of high throughput screening, the experimental conditions are adjusted to achieve a proportion of test compounds identified as “positive” compounds from amongst the total compounds screened. The assay is preferably set to identify compounds with an appreciable affinity towards the target RNA eg. when 0.1% to 1% of the total test compounds from a large compound library are shown to bind to a given target RNA with a Kᵢ of 10 μM or less (eg. 1 μM, 100 nM, 10 nM, or less).

Kits of the Invention

The invention also provides a kit for determining whether a test compound binds to a target RNA, the kit comprising the target RNA and a RNA-modifying activity.

### EXAMPLES

### Example 1

23S GAR as a Methyltransferase Substrate

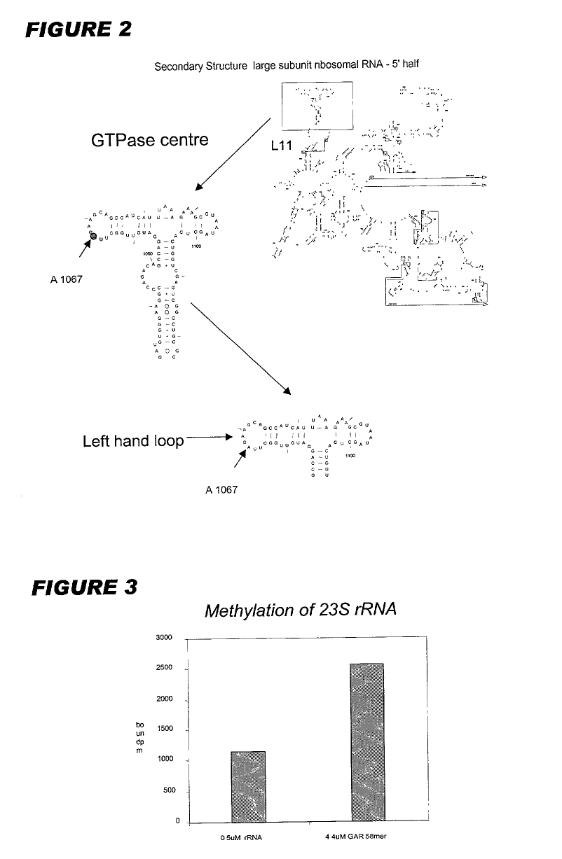

Streptomyces azureus produces a methyltransferase that introduces a single methyl group at the 2′O position of adenosine-1067 in the GTPase activating region (GAR) of its 23S rRNA (FIG. 2). The GAR is the site of action of the thiazole antibiotic thiostrepton, and the gene encoding the methyltransferase (tsr) confers resistance in Streptomyces azureus. Thiostrepton has been shown to bind a 60 mer fragment (‘mGAR’ herein) of the GAR.

The tsr gene (John Innes Foundation, Norwich) was cloned and overexpressed in E. coli. The methylation activity of the tsr methyltransferase was investigated using complete 23S E. coli rRNA and the 60 mner mGAR from Thermotoga maritima. (sequence: GGCUGGGAUGUUGGCUUAGAAGCAGCCAUCAUUUAAAGAGUGCGUAACAGCUCACCAGCC, with the methylation site underlined).

RNA (0-2 μM) was incubated in 25 mM HEPES-KOH, pH 7.5, 25 mM NH₄Cl, 5 mM MgCl₂, 5 mM DTT with test compounds. The methylation reaction was started by adding 8 pmol TSR methyltransferase and 1 μCi [³H]SAM (Amersham-Pharmacia). The final assay volume was 100 μl. The reaction was incubated 15 min at 25° C. and stopped by adding 1 vol. 2% TFA. The TFA precipitate was filtered through a 96-well glassfiber filterplate (Millipore Multiscreen FB 1.0 μM Glass Fiber Type B Filter). The filters were washed with 2 vol. of 2% TFA. 50 μl scintillation cocktail (Wallac Optiszint) was added to each well of the dried filter plate and incorporated [³H]-radioactivity was determined in a Wallac Trilux scintillation counter. As shown in FIG. 3A, 23S and mGAR were both efficient targets of the tsr product.

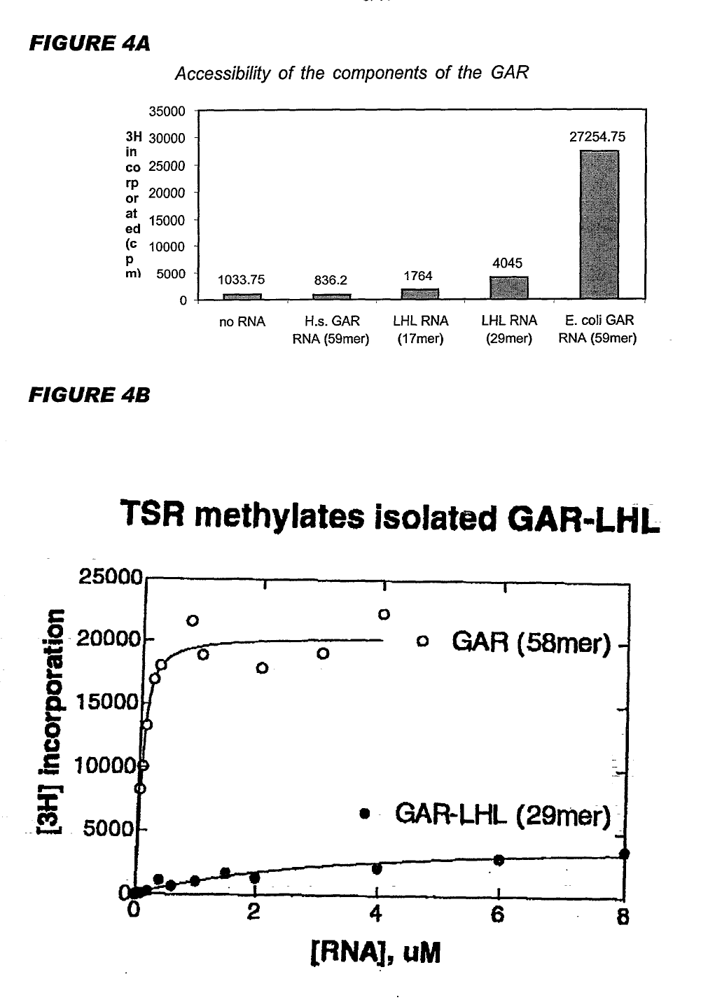

Four further RNA substrates were tested: the left-hand loop (LHL) 29 mer of E. coli mGAR (GGAUGUUGGCUUAGAAGCAGCCAUCAUCC, methylation site underlined); a 17 mer fragment of that 29 mer (GGGCUUAGAAGCAGCCU, methylation site underlined); and Homo sapiens mGAR (GGCAGGACGGUGGCCAUGGAAGUCGGAAUCCGCUAAGGAGUGUGUAACAACUCACCUGCC, the underlined residue being the homologous position to the methylation site in prokaryotes). In all bacterial RNAs, the sequences of the LHL, which contains A1067, are identical. The RNAs were incubated with TSR methyltransferase and assayed as before. As shown in FIG. 4A, the human sequence is not methylated, and the MGAR fragments are weak substrates.

The E. coli MGAR and the LHL fragment were tested as above at a variety of concentrations, and the binding curve is shown in FIG. 4B. The E. coli mGAR was a good substrate, with a Kₘ of 50 nM, compared with 2 μM for LHL. This suggests that the RNA needs to be at least partially folded to be recognised by the tsr enzyme.

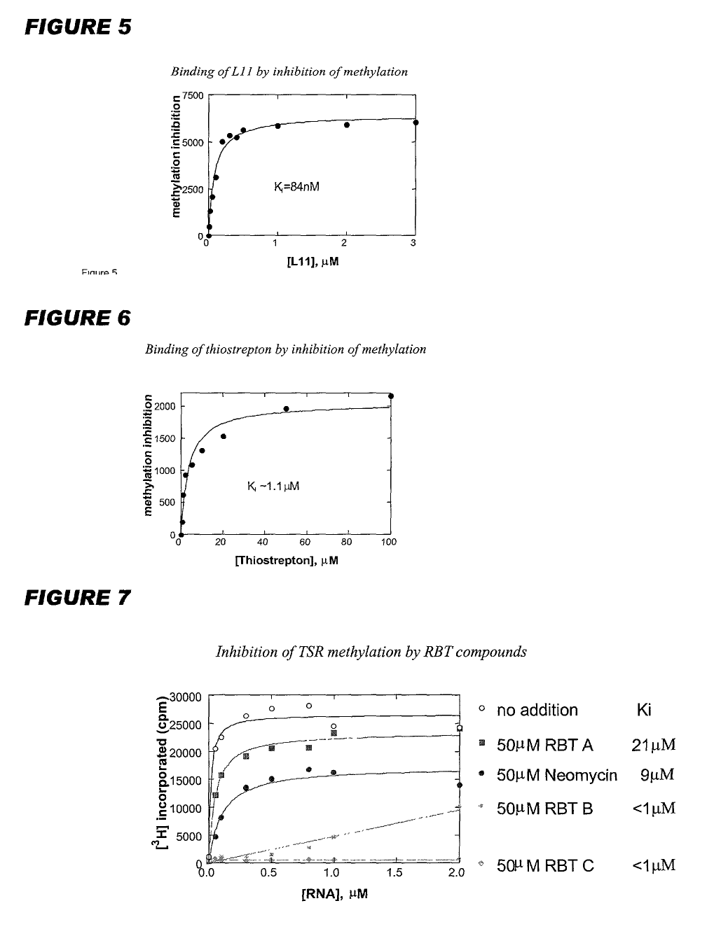

The L11 ribosomal protein is an integral part of the GAR, and its presence inhibits methyltransferase activity [Bechthold & Floss, 1994]. Various concentrations of L11 were incubated with E. coli GAR and [³H]SAM as described above. As expected, methylation was inhibited as L11 concentration increased. The affinity of the interaction (84 nM; FIG. 5) is comparable to published values.

As discussed above, thiostrepton does not bind to 23S which is methylated at A-1067. Methyltransferase and thiostrepton thus compete for binding at A-1067, and binding of thiostiepton to 23S (or GAR) can be measured by following inhibition of methylation. FIG. 6 shows the result of this experiment, which gave an affinity of 1.1 μM, comparable to published values.

### Example 2

Identifying RNA-binding Compounds

The tsr methyltransferase product is thus an intimate probe of intermolecular contacts in the GAR, and can measure interactions specifically at positions where antibiotics interact. Compounds which inhibit the methylation (and thus inhibit antibiotic resistance) can therefore be identified using this assay.

Test compounds were assayed in a 96 well plate for inhibition of tsr methylation activity at 50 μM. Compounds were incubated at 25° C. for 30 minutes in the presence of [³H]SAM and enzyme, and increasing amounts of mGAR were added. RNA was precipitated with 2% TFA, filtered, and scintillation counted in a Wallac Trilux instrument.

FIG. 7 shows the results of this experiment using neomycin, RBT-A, RBT-B and RBT-C as test compounds. It is clear that all compounds show significant inhibition of methyltransferase, consistent with the Kᵢ values shown in the figure. RBT-C shows very good inhibition of methylation.

### Example 3

Further Measurement of Interactions at the GTPase Center

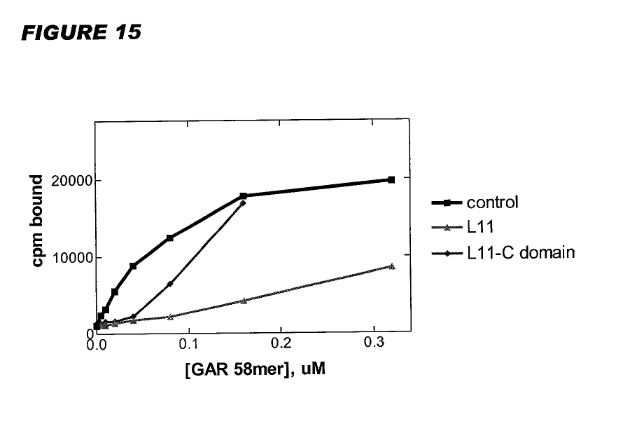

Binding of the ribosomal protein L11 inhibits methyltransferase activity (FIG. 5). Methyltransferase activity can be recovered by deletion of the N-terminal domain of L11 (FIG. 15). It is therefore possible to modify the assay such that fragments of proteins are incorporated.

The concentration of L11 and L11-C-domain was 1 μM. The assay was performed in 25 mM HEPES/KOH, pH 7.5, 5 mM MgCl₂, 25 mM NH₄Cl, 5 mM DTT. GST-TSR methylase (100 nM final conc.) and 0.05 μCi of [³H]SAM (Amersham Pharmacia Biotech) were added in a final volume of 100 μl. GAR domain RNA transcript was titrated from 0-320 nM. The assay was incubated for 30 min at 25° C. in wells of a Multiscreen 96-well glass fiber filter plate (Millipore). The assay was stopped by precipitating with 1 vol. of 2% TFA and washing twice with 2 vol. of 2% TFA. After drying of the filters, 40 μl of Optiphase Supermix scintillation liquid (PerkinElmer) were added per well and the incorporated radioactivity was counted in a Trilux scintillation counter (PerkinElmer).

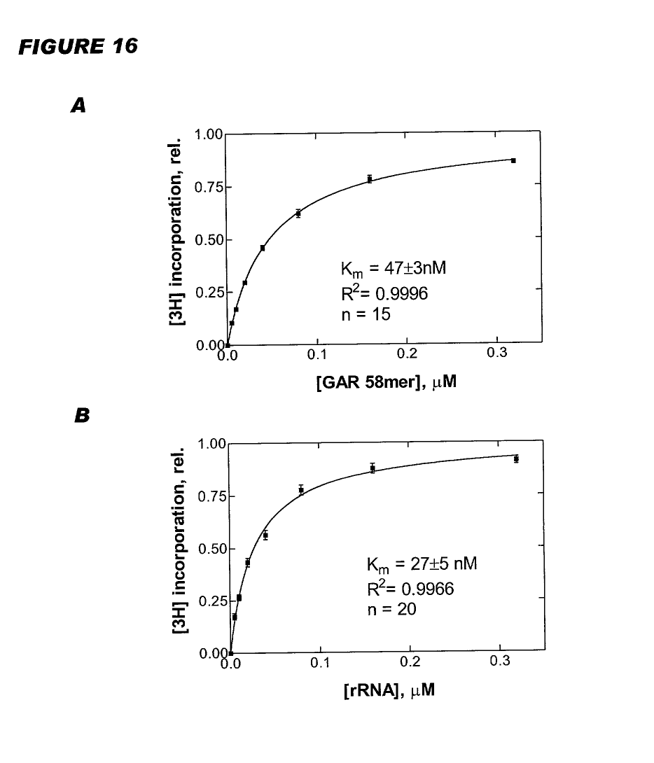

The enzyme is also active on ribosomal RNA (rRNA) as shown in FIG. 16.

### Example 4

Reproducibility of the Assay

The Methyltransferase activity of the TSR methyltransferase assay is highly consistent, in experiments where either ribosomal RNA or GAR 58 mer RNA are methylated, the Kₘ can be measured reproducibly. Data obtained were fitted to the Michaelis-Menten equation by non-linear least-square fitting using GraphPad Prism software. Error bars represent the standard error of means, the KₘR² and number of experiments are indicated (FIG. 16). The Kₘ measured for rRNA over 20 experiments is 27 (+/−5) nM. The Kₘ for GAR 58 mer RNA over 15 experiments is 47(+/−3) nM (FIG. 16).

FIG. 16 shows data from fifteen independent methylation reactions of Gar 58 mer, presented as titrations. FIG. 16B shows data from twenty independent methylation reactions of rRNA, presented as titrations.

All assays were performed in 96-well format in 25 mM HEPES/KOH, pH 7.5, 5 mM MgCl₂, 25 mM NH₄Cl, 5 mM DTT. GST-TSR methylase (100 nM final conc.) and 0.05 μCi of [³H]SAM (Amersham Pharmacia Biotech) were added in a final volume of 100 μl. GAR domain RNA transcript was titrated from 0-320 nM. The assay was incubated for 30 min at 25° C. in wells of a Multiscreen 96-well glass fiber filter plate (Millipore). The assay was stopped by precipitating with 1 vol. of 2% TFA and washing twice with 2 vol. of 2% TFA. After drying of the filters, 40 μl of Optiphase Supermix scintillation liquid (PerkinElmer) were added per well and the incorporated radioactivity was counted in a Trilux scintillation counter (PerkinElmer).

### Example 5

Single Point Assays

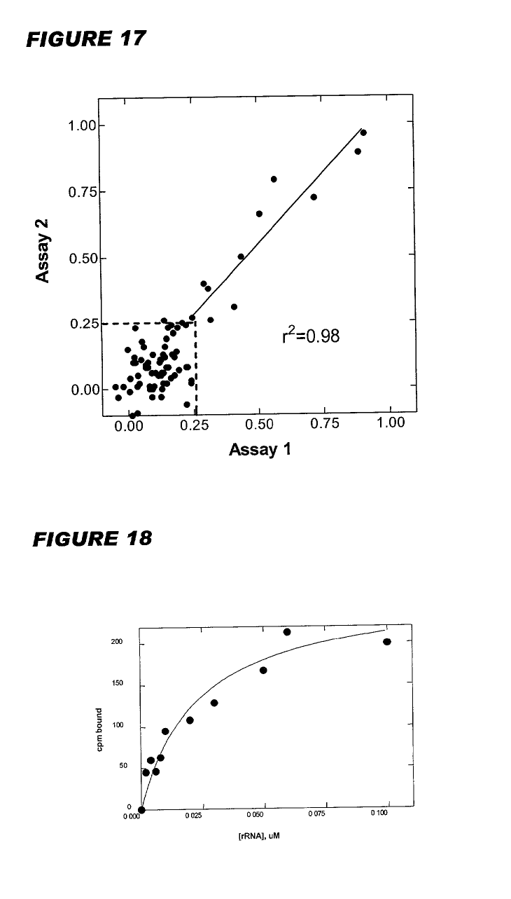

The assay can be implemented in a single point format. The consistency and reproducibility of this assay format is shown in FIG. 17. The same set of 80 compounds were assayed in single points at 50 μM. The activities of the compounds in the two assays were plotted against each other. Active compounds (those that show greater than 25% inhibition) lie close to the diagonal, demonstrating the reproducibility of the single point assay format.

Activities are given as % inhibition compared to the control in the absence of compound. The correlation analysis for active compounds (showing >25% inhibition) yields a r²=98. All assays were performed in 96-well format in 25 mM HEPES/KOH, pH 7.5, 5 mM MgCl₂, 25 mM NH₄Cl, 5 mM DTT. GST-TSR methylase (100 nM final conc.) and 0.05 μCi of [³]SAM (Amersham Pharmacia Biotech) were added in a final volume of 100 μl, the GAR domain RNA concentration was 25 nM. The assay was incubated for 30 min at 25° C. in wells of a Multiscreen 96-well glass fiber filter plate (Millipore). The assay was stopped by precipitating with 1 vol. of 2% TFA and washing twice with 2 vol. of 2% TFA. After drying of the filters, 40 μl of Optiphase Supermix scintillation liquid (PerkinElmer) were added per well and the incorporated radioactivity was counted in a Trilux scintillation counter (PerkinElmer).

### Example 6

Measurement of Erythromycin Resistance Methyltransferase Activity

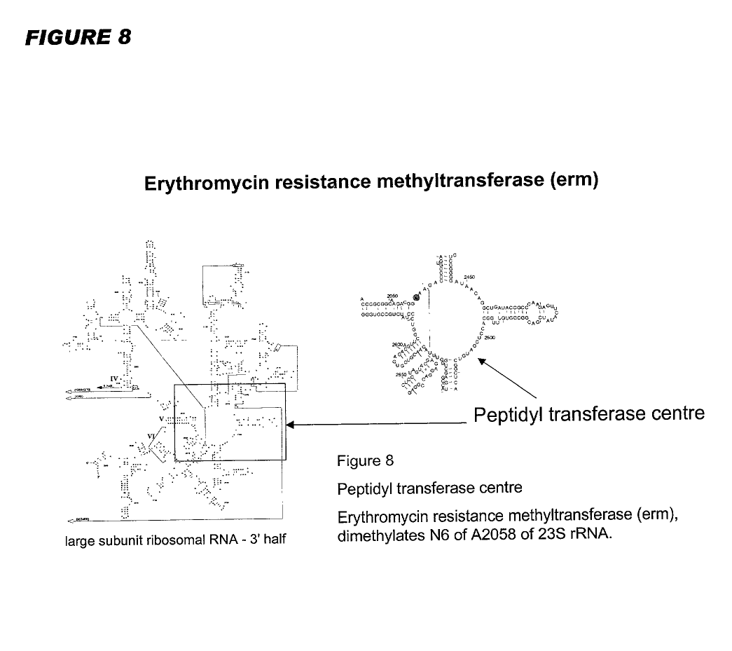

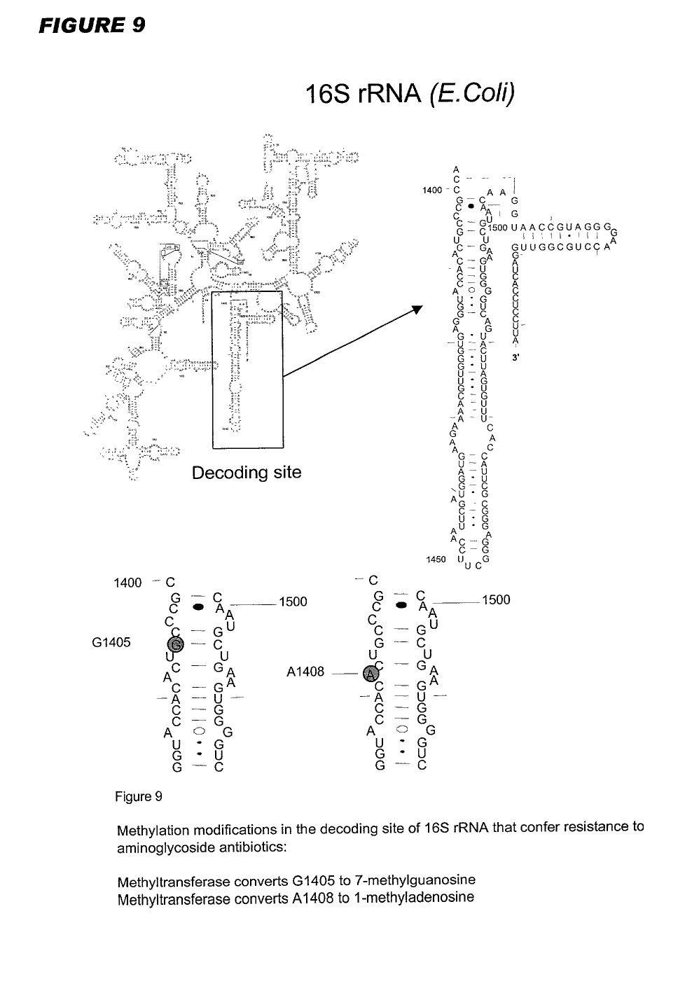

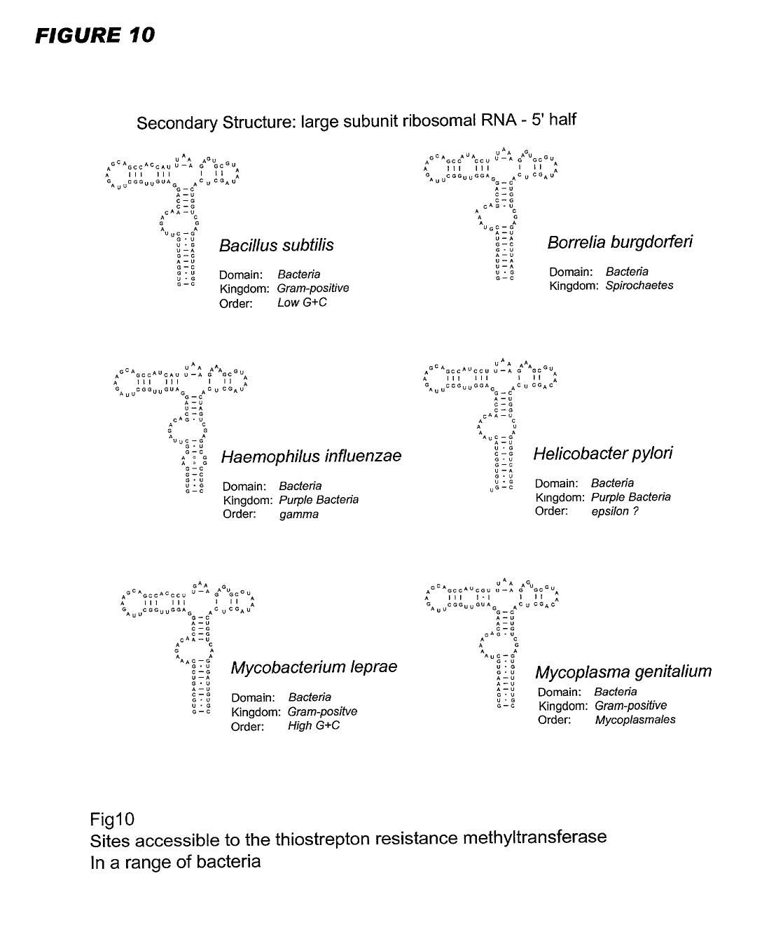

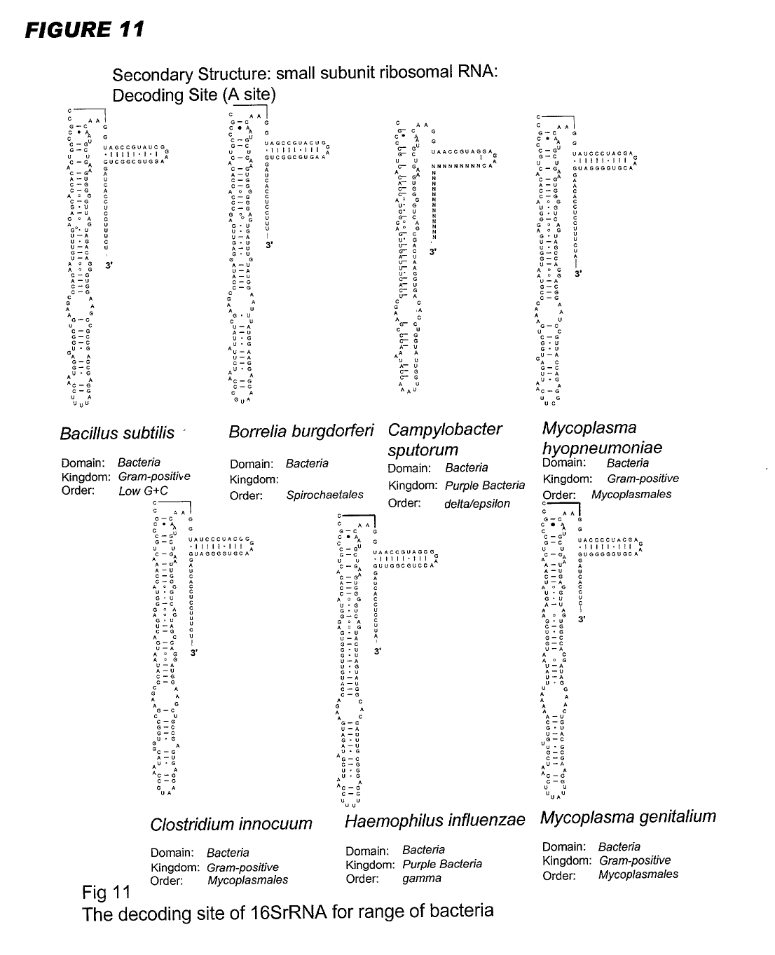

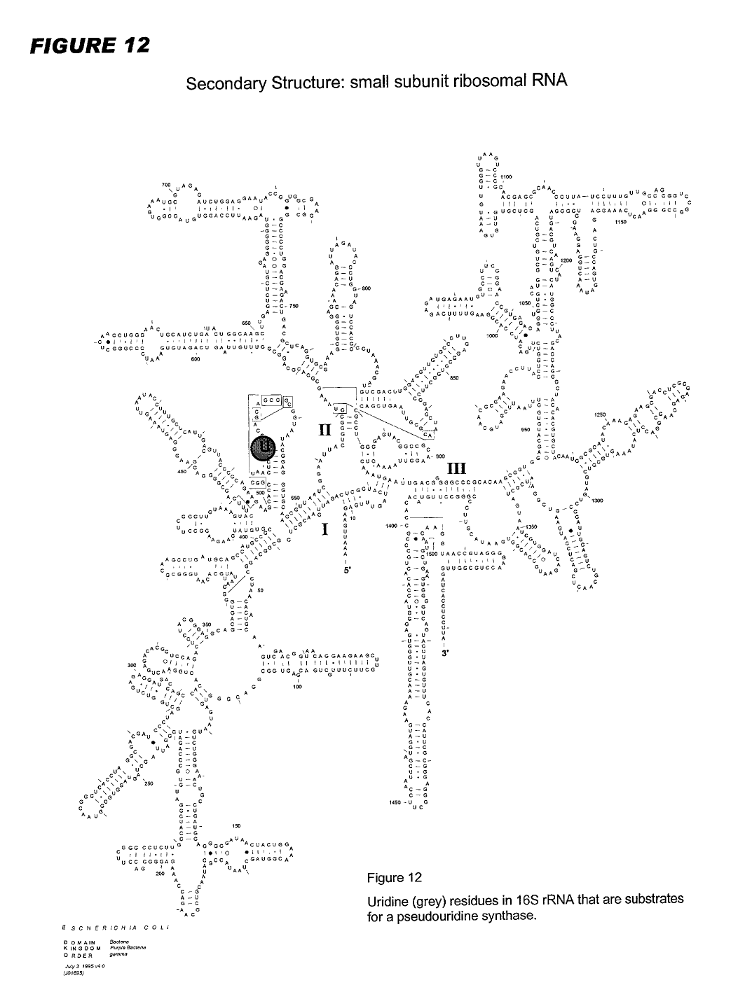

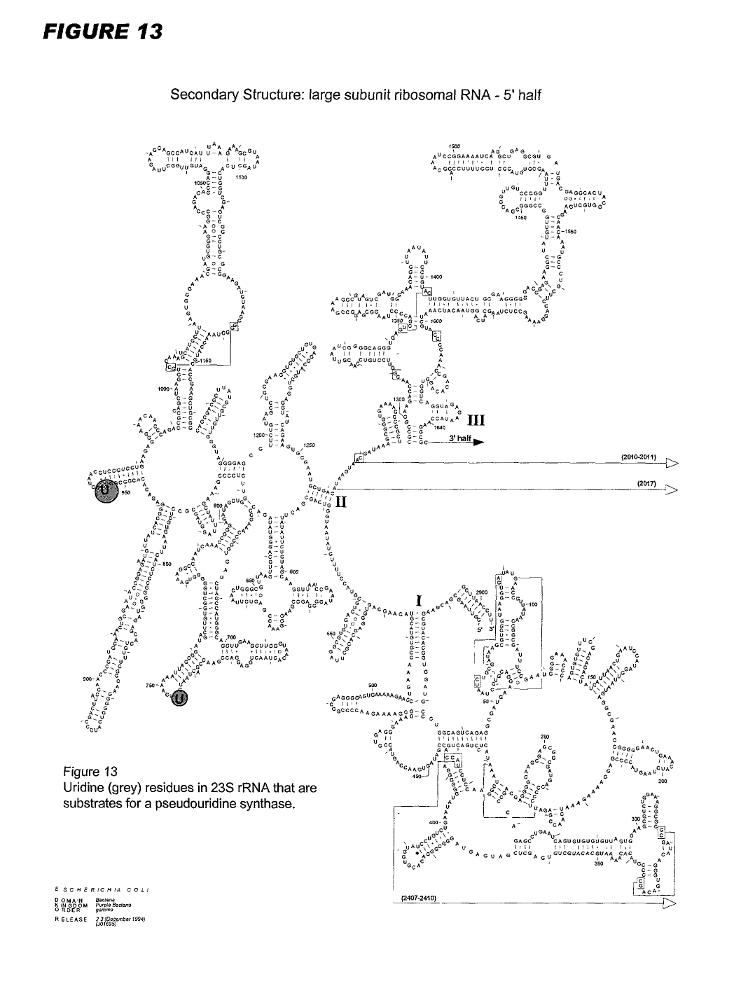

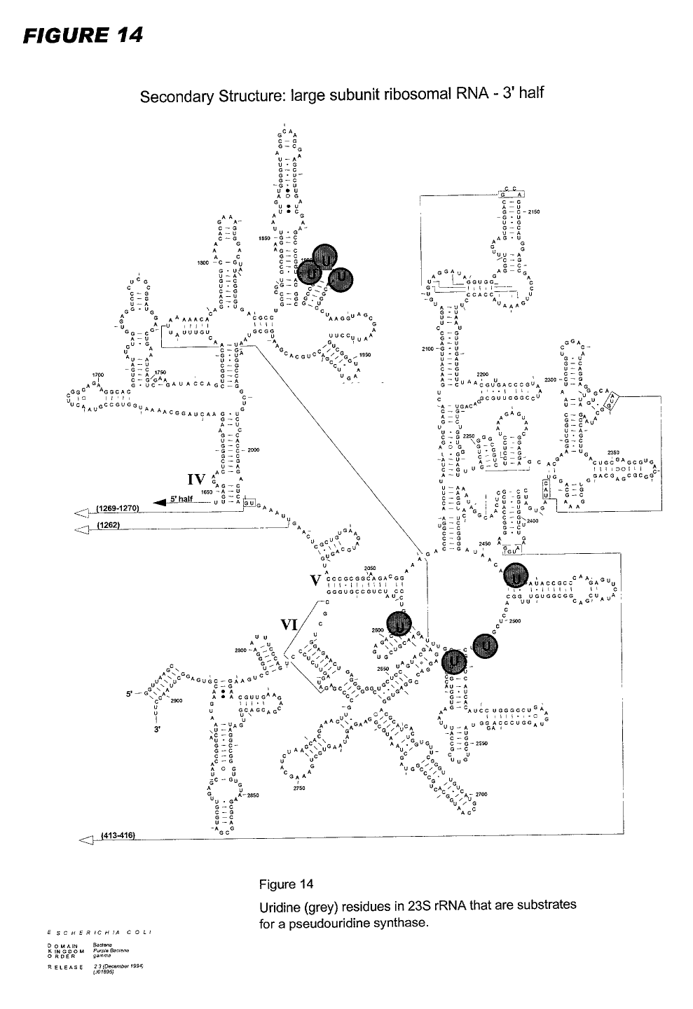

The erythromycin resistance methyltransferases are a class of methyltransferases (Erm) that confer resistance to the macrolide class of antibiotics, exemplified by erythromycin, modification occurs in the peptidyl transferase centre of 23S rRNA (FIG. 8). ErmE incorporates methyl groups into 23S rRNA (FIG. 18) with a Km of 23 nM. The reaction was performed in 100 μl 20 mM HEPES/KOH, pH 7.8, 10 mM MgCl₂, 100 mM NH4Cl, 1 mM DTT, 10% glycerol for 30 min at 25° C. with 0.01 μM GST-Erm methylase and 0.5 μCi [3H]SAM. The Km derived from the data shown is 23 nM. Assays were performed in 96-well format in 25 mM HEPES/KOH, pH 7.5, 5 mM MgCl₂, 25 mM NH₄Cl, 5 mM DTT. ErmE methylase (100 nM final conc.) and 0.05 μCi of [³H]SAM (Amersham Pharmacia Biotech) were added in a final volume of 100 μl. GAR domain RNA transcript was titrated from 0-320 nM. The assay was incubated for 30 min at 25° C. in wells of a Multiscreen 96-well glass fiber filter plate (Millipore). The assay was stopped by precipitating with 1 vol. of 2% TFA and washing twice with 2 vol. of 2% TFA. After drying of the filters, 40 μl of Optiphase Supermix scintillation liquid (PerkinElmer) were added per well and the incorporated radioactivity was counted in a Trilux scintillation counter (PerkinElmer).

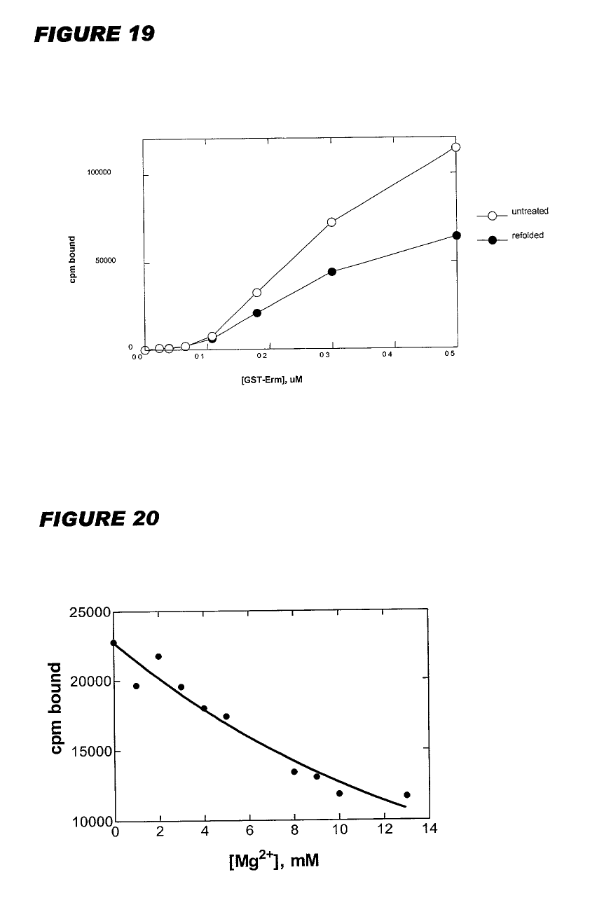

The methylation activity of the methytransferase is dependent on the conformation of the RNA (FIG. 19), and significantly less radioactivity is incorporated into rRNA that has undergone a folding/hybridisation step. FIG. 19 shows the effect of refolding on rRNA methylation by GST-Erm. The reaction was performed in 50 μl 50 mM Tris/Cl, pH 7.5, 4 mM MgCl₂, 40 mM KCl, 10 mM DTT, for 120 min at 37° C. with 0.04 μM rRNA and 6 μCi [3H]SAM. The rRNA as either untreated or folded by heating 3 min at 65° C. and then stored on ice.

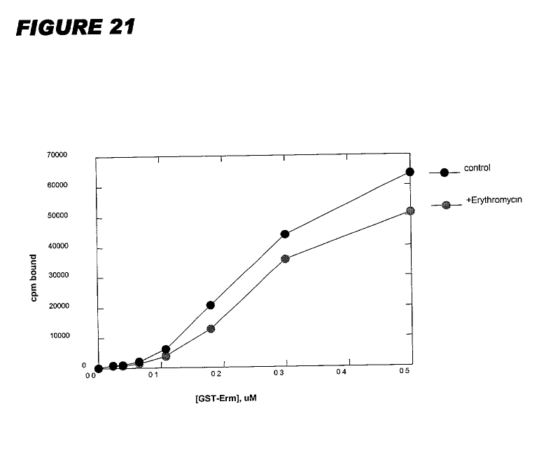

To characterise the magnesium dependence of the Erm E methyltransferase, the incorporation of radioactivity into ribosomal RNA was measured over a range of magnesium concentrations (FIG. 20). The stringency of the assay may be altered by adjusting the magnesium concentration in the assay. The assay was performed in 50 mM Tris/Cl, pH 7.5, 40 mM KCl, 10 mM DTT. For refolding, rRNA (Roche) was incubated 3 min at 65° C., cooled down to room temperature over 3 h, then put on ice and stored at −20° C. After refolding, Magnesium chloride (0-14 mM), GST-ERM methylase (200 nM final conc.) and 0.12 μCi of [³H]SAM (Amersham Pharmacia Biotech) were added in a final volume of 50 μl. The final rRNA concentration was 170 nM. The assay was incubated for 2 h at 37° C. in wells of a Multiscreen 96-well glass fibre filter plate (Millipore). The assay was stopped by precipitating with 1 vol. of 2% TFA and washing twice with 2 vol. of 2% TFA. After drying of the filters, 40 μl of Optiphase Supermix scintillation liquid (PerkinElmer) were added per well and the incorporated radioactivity was counted in a Trilux scintillation counter (PerkinElmer).

### Example 7

Inhibition of Erythromycin Resistance Methylase ermE by Erythromycin.

Erm E is inhibited by erythromycin (FIG. 21). Erythromycin inhibits enzyme activity most noticably when the RNA is folded in the presence of erythromycin (FIG. 21). One embodiment of the invention may therefore involve folding of the RNA in the presence of compound before measuring the activity of the methyltransferase on the RNA.

FIG. 21 shows the decrease in refolded rRNA methylation by Erm E in the presence of erythromyin. The reaction was performed in 50 μl 50 mM Tris/Cl, pH 7.5, 4 mM MgCl₂, 40 mM KCl, 10 mM DTT, for 120 min at 37° C. with 0.04 μM rRNA and 6 μCi [3H]SAM. The erythromycin concentration was 200 μM. Erythromycin showed no effect when used with unfolded rRNA.

### Example 8

Inhibition of Erythromycin Resistance Methylase ermE by Test Compounds in a Single Point Assay Format

A series of compounds A-J were assayed at 50 μM, compounds C-G and J showed significant (greater than 40%) inhibition of the methyltransferase. The ermE assay can therefore be implemented in a high throughput screening format.

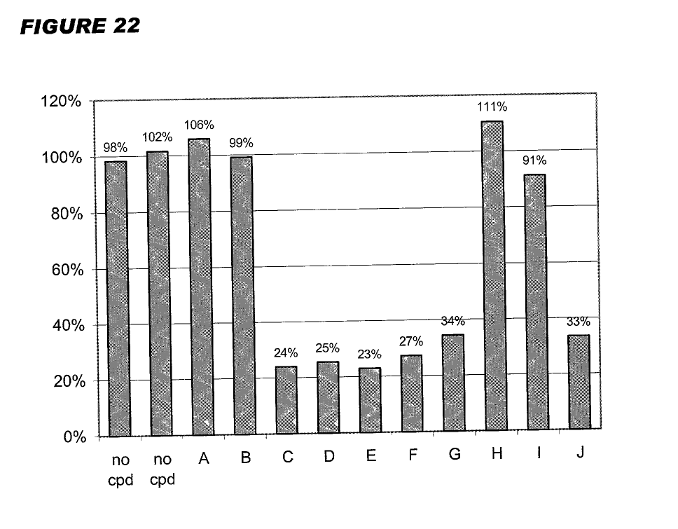

FIG. 22 shows inhibition of rRNA methylation with ERM by various compounds. The identity is unimportant as the present example merely serves to demonstrate the utility of a single point assay format. The assay was performed as above, except that the MgCl₂ concentration was 10 mM and the final rRNA concentration was 40 nM. Compounds A-J were assayed at 50 μM. The final DMSO concentration was 1%. The activity is given relative to the average activity in the absence of compound.

It will be understood that the invention has been described by way of example only and modifications can be made whilst remaining within the scope and spirit of the invention.

1. A method for determining whether a test compound binds to a target RNA, the method comprising the steps of:
(a) contacting said test compound with said target RNA and a RNA-modifying enzyme; and
(b) detecting the modification of said target RNA by said enzyme and comparing the amount of modification detected to that of a standard, wherein said comparing determines whether said test compound binds to said target RNA.

2. The method of claim 1, wherein said target RNA comprises a rRNA or a fragment or sub-region thereof.

3. The method of claim 1 wherein said target RNA comprises a whole ribosome.

4. The method of claim 1 wherein said target RNA is a ribosome fragment or sub-region thereof.

5. The method of claim 1, wherein said target RNA includes a stabilising structure.

6. The method of claim 1, wherein said target RNA comprises a chemical modification which enhances the stability of said target RNA.

7. The method of claim 1, wherein said RNA-modifying enzyme is selected from the group consisting of a methyltransferase, a pseudouridine synthase, a guanine glycosylase, a G37-N1-methylguanosine-tRNA-methyltransferase, and a 2′-O-ribosyl phosphate transferase.

8. The method of claim 7, wherein said methytransferase is the thiostrepton resistance methyltransferase or the erythromycin resistance methyltransferase.

9. The method of claim 7, wherein target RNA modification is detected by the incorporation of an isotopic label from S-adenosyl-methionine into said target RNA.

10. The method of claim 8, wherein target RNA modification is detected by the incorporation of an isotopic label from S-adenosyl-methionine into said target RNA.

11. The method of claim 1, wherein said test compound is selected from the group consisting of a peptide, a peptoid, a protein, a lipid, a metal, a nucleotide, a nucleoside, a small organic molecule, and a polyamine.

12. The method of claim 1, wherein said test compound is selected from a combinatorial library.

13. The method of claim 1, in a high-throughput screening format.

14. A compound with antibiotic activity that binds to a target RNA, said compound identified by
(a) contacting a test compound with said target RNA and a RNA-modifying enzyme; and
(b) detecting the modification of said target RNA by said enzyme and comparing the amount of modification detected to that of a standard, wherein reduced modification of said target RNA in the presence of said test compound identifies said test compound as a compound with antibiotic activity.

15. A kit for determining whether a test compound binds to a target RNA, said kit comprising said target RNA and a RNA-modifying enzyme.

16. A method for determining whether a test compound binds to a target RNA, said method comprising the steps of:
(a) contacting said test compound with a RNA-modifying enzyme and said target RNA, wherein said target RNA comprises a suicide substrate for said enzyme; and
(b) detecting the modification of the enzyme by said suicide substrate, wherein said detecting determines whether said test compound binds to said target RNA.
